(19) United States
(12) Patent Application Publication
(10) Pub. No.: US 2004/0156882 A1
(43) Pub. Date: Aug. 12, 2004

(54) Method and composition for feeding mammals

(76) Inventors: David F. Davenport, Knoxville, TN (US); J. Eric Martin, Louisville, TN (US)

Correspondence Address: ST. ONGE STEWARD JOHNSTON & REENS, LLC, 986 BEDFORD STREET, STAMFORD, CT, 06905-5619, US

(21) Appl. No.: 10/692,063

(22) Filed: Oct. 23, 2003

Related U.S. Application Data
(60) Provisional application No. 60/420,548, filed on Oct. 23, 2002.

(51) Int. Cl. A61K 47/00

(52) U.S. Cl. 424/439

(57) ABSTRACT

Provided is a nutraceutical composition having a high energy, low fat component, suitable for enteric administration, present in an effective proportion such that upon administration to a mammal in need thereof, the nutraceutical composition is effective to improve absorption of nutrients, increase appetite, promote weight gain, or reduce calorie deficit. A method for reducing energy deficit in a mammal comprising the step of enterically administering to the mammal energy promoting effective amount of a nutraceutical composition comprising a low fat component in an effective proportion is also provided.

Applicants claim priority benefits under 35 U.S.C. §119(e) of U.S. Provisional Patent Application Serial No. 60/420,548 filed Oct. 23, 2002.

### FIELD OF THE INVENTION

The present invention relates to therapeutic compositions and methods for promoting nutrition in mammals in need thereof and, in particular to compositions capable of promoting recovery of a diseased or injured mammal such as a horse. Furthermore, the invention relates to a nutraceutical composition, which is useful for weight gain for hypophagic or dysphagic subjects.

### BACKGROUND OF THE INVENTION

Hypophagic or dysphagic horses can benefit from enteral nutrition. However, conditions such as colitis, enteritis, hepatic disease, renal disease, neoplasia and post-operative recovery require different nutritional considerations. Catabolic states that result from disease or surgical procedures often reduce the prognosis for recovery or unnecessarily lengthen the recovery period. Customized nutritional support not only provides calories in the form of carbohydrates, fats and proteins, but it can address specific nutritional needs and help alter the disease process to promote healing.

Currently practitioners depend largely on ground pelleted feeds when enteral nutrition is deemed necessary. Pelleted feeds are able to provide adequate calories but fall short of providing specific nutrients that may be beneficial in certain patients, or even provide an overabundance of a detrimental nutrient. Human enteral formulas have been used with some degree of success. However, these diets fall short in dietary fiber, are very high in fat and due to the volume of formula used are often cost-prohibitive. A low fat feeding program provides much more flexibility to the practitioner in terms of devising a strategic approach to feeding horses in different disease conditions. This can be achieved in a simple and cost-effective manner.

Thus, there is a need for a composition having low fat content which can provide highly concentrated, highly digestible nutrients in a both flexible and convenient manner to a mammal in need of nutrients.

Enteral nutrition is often required by horses experiencing severe disease. It is possible to provide nutrition in the form of slurry feeds through nasogastric or esophagostomy tubes. By providing a novel critical care enteral feeding program low in fat, practitioners can target specific needs and metabolic conditions. Providing a low fat nutraceutical composition has been found to improve the long-term prognosis for recovery and can be done both simply and cost effectively.

There remains a need for nutritional supplements for mammals such as a human or horse which can improve health when administered in an enteral dosage to an animal in need of nutrients. This improvement can be, for example, improved weight gain, bone strength, the reduction of energy deficit, or the infusion of energy in a low fat formulation. The present invention provides the medical and veterinarian practitioner with easy and cost-effective formulizations of a component based critical care feeding program that can be adjusted for a subject's needs.

### SUMMARY OF THE INVENTION

It is an object of the present invention to provide compositions that can be administered to a subject through an enteral feeding tube.

It is an object of the present invention to provide palatable compositions for mammals.

It is an object of the present invention to provide economical or inexpensive supplements for mammals.

It is an object of the present invention to administer compositions having a high concentration of nutrient through an enteral feeding tube to a mammal in need thereof.

It is an object of the present invention to provide compositions suitable for administration that crosses life stages and/or species.

It is an object of the present invention to provide a method that will allow easy and cost-effective formulization of a component based critical care feeding program pre-adjusted for a subjects needs.

It is an object of the present invention to provide energy to mammals in need thereof.

These and other objectives of the present invention are obtained by providing a composition suitable for administration through an enteral feeding tube comprising an effective proportion such that upon administration to a mammal in need thereof, the composition is effective to improve absorption of nutrients, increase appetite, promote weight gain, or reduce calorie deficit, wherein the composition comprises less than 3% by weight fat. Optionally, in some embodiments the composition may be further characterized as having between about 2% to about 2.5% fat by weight. Optionally, and in some embodiments the composition comprises a feed component comprising ingredients selected from the group consisting of alfalfa, corn meal, oats, and combinations of these. Optionally, and in some embodiments the composition further comprises a liquid vitamin. Optionally, and in some embodiments the composition comprises a nutrient component comprising at least one vitamin selected from the group consisting of vitamin A, vitamin B-1, vitamin B-2, vitamin B-3, vitamin B-6, vitamin B-12, vitamin C, vitamin D-3, vitamin E, vitamin K, biotin, choline, folic acid, and combinations of these. Optionally, and in some embodiments the composition comprises a nutrient component comprising at least one mineral selected from the group consisting of calcium, magnesium, potassium, boron, molybdenum, vanadium and combinations of these. Optionally, the nutrient component comprises at least one trace mineral selected from the group consisting of iron, copper, zinc, manganese, chromium, iodine, selenium, and combinations thereof. Optionally, the nutrient component comprises at least one antioxidant selected from the group consisting of CoQ10, pantothenic acid, DMG, grape seed extract, bioflavinoid, inositol, PABA, citrus bioflavonoid, pyctogen, and combinations of these. Optionally, the composition comprises a nutrient component comprising at least one amino acid selected from the group consisting of alanine, arginine, aspartic acid, cystine, glutamic acid, proline, glycine, histidine, hydroxyproline, isolcucine, leucine, lysine, methionine, phenylalanine, serine, threonine, tryptophan, tyrosine, valine, and combinations thereof. The composition may further comprise whey powder. In such compositions comprising whey powder the whey powder is smaller than about 45 mesh. Optionally the composition comprises a protein component comprising the following constituents: whey powder, and lactase. In such embodiments, the constituents of the protein component are present in the following approximate effective proportions: between about 95 and about 100% by weight of whey powder, and between about 1% to about 5% by weight of lactase. Such an embodiment may further comprise a protein component further comprising at least one monosaccharide. Moreover, in such a composition the monosaccharide may be selected from the group consisting of glucose, galactose, fructose, and combinations of these.

Where compositions comprise a nutrient component, the nutrient component optionally may further comprise an ingredient selected from the group consisting of vitamin, mineral, trace mineral, antioxidant, amino acid and combinations of these. Moreover, such compositions may further comprise a mineral or trace mineral is in amino acid chelate form.

In compositions comprising amino acids, the amino acid may be selected from the group consisting of L-glutamine, L-arginine, carnitine, and combinations of these.

In compositions comprising a functional food component, the functional food component comprises an ingredient selected from the group consisting of glucosamine, salt, amino acid, yeast, fermentation extract, and combinations these ingredient. Where such composition uses glucosamine, the glucosamine is a chemical selected from the group consisting of glucosamine sulphate, glucosamine sulfate 2KCL, glucosamine sulfate NaCl, glucosamine hydrochloride, N-acetylglucosamine, Poly-Nag. glucosamine, and combinations of these. Where such composition uses salt, sodium chloride is the preferred salt. Where such a composition uses a fermentation extract, the fermentation extract may comprise a comprise a prebiotic, probiotic, synbiotic, or combinations of these.

In some embodiments, the composition comprises the following components: a protein component, a nutrient component, a functional food component, and a feed component, wherein each component is present in an effective proportion such that, when administered to a mammal in need thereof in an effective amount, the nutraceutical composition is effective to improve absorption of nutrients. Where such a composition is provided, the composition may be in a liquid dosage form.

The objectives of the present invention are further obtained by providing a method for reducing energy deficit in a mammal comprising the step of enterically administering to the mammal energy promoting effective amount of a composition comprising an effective proportion having less than 3% fat. Such a method may optionally provide a composition comprising between about 2% to about 2.5% fat by weight. Such a method may optionally provide a composition wherein the composition comprises a nutrient component further comprising ingredients selected from the group consisting of vitamin, mineral, trace mineral, antioxidant, amino acid and combinations thereof. Some embodiments optionally include a method wherein the composition comprises a nutrient component further comprising liquid vitamin. Such a method may optionally include a composition comprising a nutrient component comprising at least one vitamin selected from the group consisting of vitamin A, vitamin B-1, vitamin B-2, vitamin B-3, vitamin B-6, vitamin B-12, vitamin C, vitamin D-3, vitamin E, vitamin K, biotin, choline, folic acid, and combinations thereof. Such a method may further optionally include a composition comprising a nutrient component comprising at least one antioxidant selected from the group consisting of CoQ10, pantothenic acid, DMG, grape seed extract, bioflavinoid, inositol, PABA, citrus bioflavonoid, pyctogen, and combinations of these. Such a method may optionally provide a composition comprising a feed component further comprising ingredients selected from the group consisting of alfalfa, oats, and combinations thereof. Such a method may further optionally provide a composition is in an oral liquid dosage form, or a powder form.

Such methods are suitable for use on mammals, including but not limited to, a human, horse, dog, cow, pig, goat, or sheep.

In some embodiments, the method optionally further provides a composition comprising a nutrient component comprising at least one mineral selected from the group consisting of calcium, magnesium, potassium, boron, molybdenum, vanadium and combinations thereof. Such a method may optionally include mineral in amino acid chelate form.

In some preferred embodiments, the method optionally includes a composition comprising a nutrient component comprising at least one trace mineral selected from the group consisting of iron, copper, zinc, manganese, chromium, iodine, selenium, and combinations of these. Such a method preferably includes mineral is in amino acid chelate form.

In certain preferred embodiments, the method may provide a composition comprising ingredients selected from the group consisting of whey powder, lactase, and combinations thereof. Such methods may optionally include a composition comprising whey powder and lactase, wherein the whey powder and lactase are present in the following approximate effective proportions: between about 95% to about 100% by weight of whey powder, and between about 1% to about 5% by weight of lactase. Such methods preferably include whey powder smaller than about 45 mesh. Such a composition may further have a protein component. Optionally the composition may further comprise at least one monosaccharide. Suitable monosaccharide may be selected from the group consisting of glucose, galactose, fructose, and combinations thereof.

In some preferred embodiments, the method provides a composition comprising a nutrient component. That nutrient component may comprise at least one amino acid selected from the group consisting of alanine, arginine, aspartic acid, cystine, glutamic acid, proline, glycine, histidine, hydroxyproline, isolcucine, leucine, lysine, methionine, phenylalanine, serine, threonine, tryptophan, tyrosine, valine, and combinations thereof.

Some preferred embodiments may also include a method wherein the composition comprises a functional food component further comprising ingredients selected from the group consisting of glucosamine, salt, amino acid, yeast, fermentation extract, and combinations thereof. In such embodiments, the glucosamine is a chemical selected from the group consisting of glucosamine sulphate, glucosamine sulfate 2KCL, glucosamine sulfate NaCl, glucosamine hydrochloride, N-acetylglucosamine, Poly-Nag. glucosamine, and combinations thereof. In such an embodiment, the salt is preferably sodium chloride. In such an embodiment the amino acid is preferably selected from the group consisting of L-glutamine, L-arginine, carnitine, and combinations of these. In such an embodiment, the fermentation extract preferably comprises an ingredient selected from the group consisting of prebiotic, probiotic, synbiotic, and combinations thereof.

### Definition of Terms

The following definitions apply throughout the present specification:

The term “nutrient” refers to any substance that furnishes nourishment to an animal. The term further refers to substances such as protein, fat, carbohydrate, simple sugar, functional food, vitamin, mineral, trace mineral, antioxidant, prebiotic, probiotic, synbiotic, acid, base, or salt that provides nourishment to an animal. The term further refers to complexes of protein, fat, carbohydrate, simple sugar, vitamin, mineral, trace mineral, prebiotic, probiotic, synbiotic, acid, base, or salt that provides nourishment to an animal.

The term “probiotic” refers to a substance or organism which contributes to intestinal microbial balance in an animal. The term further refers to living organisms in foods and feeds or dietary supplements which contribute to intestinal microbial balance in an animal.

The term “prebiotic” refers to a substance or ingredient that when provided to the digestive tract selectively supports the growth of beneficial bacterial species over pathogenic ones. The term further refers to substances that do not directly colonize the digestive tract. The term prebiotic further refers to, but is by no means limited to yeast, yeast cultures, fungal cultures, and preferably, certain fibers (FOS-fructooligosaccharides).

The term “synbiotic” refers to substances or ingredients that contain both prebiotic and probiotic ingredients. The term further refers to a prebiotic and probiotic blend for gastrointestinal support.

The term “functional food” refers to a food which contains one or a combination of components which affects functions in the body so as to have positive cellular or physiological effects. The term further refers to prebiotic(s), probiotic(s), and/or synbiotic(s).

The term “parts by weight”, abbreviated “pbw”, is given its usual and customary meaning wherein a part can be expressed with reference to any convenient unit of measure, for example ounce or gram. When used with respect to a component or constituent, pbw is with reference to the total nutraceutical composition. For components or constituents that can include water of crystallization (hydration), pbw are based on the component or constituent in the non-hydrated form. The term “pbw” refers to a mix ratio as parts by weight. The term refers generally to mixing by weight.

As used herein and unless otherwise specified, percent (%) refers to percent-by-weight. For components or constituents that can include water (hydration), percentages are based on the component or constituent in the non-hydrated form.

The term “mammal” refers to any of a class (Mammalia) of warm-blooded higher vertebrates (as placentals, marsupials, or monotremes) that nourish their young with milk secreted by mammary glands, have the skin usually more or less covered with hair, and include humans. The term further refers to domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for human or veterinary medical use.

### DETAILED DESCRIPTION

The composition of the present invention includes a low fat component. According to the principles of the present invention, a predetermined composition of low fat component is administered to a mammal in need thereof. The composition is provided in an effective proportion so as to be available for use by the mammal's metabolic processes, and thus is useful in the prophylaxis or treatment, for example, of conditions or diseases in which enhanced absorption of nutrient, weight gain, removal of an energy deficit or an increased appetite are desirable in a mammal, among other desirable activities. The composition of the present invention provides a formulation having an overall fat content (% weight) of less than 3%, preferably between about 2% to about 2.5%, and most preferably below 2.5%. Using relatively simplistic ingredients, the composition of the present invention provide formulations requiring little to no digestion or metabolism for the nutrients to be utilized by the body of an individual in need thereof. The compositions deliver effective proportions of macro nutrients to mammals.

Effective proportion means at least the amount of a component or constituent necessary or sufficient so that, when administered, a nutraceutical composition that includes effective proportions of all components or constituents is effective to enhance absorption of nutrients, promote weight gain, remove energy deficit or to increased appetite.

The commercially available components and constituents useful in the practice of the present invention can be used as supplied in pharmaceutically acceptable purity. A reference to a substance includes the essentially pure substance, as well as the substance having the kinds and amounts of impurities as the skilled artisan knows or expects to be present in the commercially available substance.

When each of the components and other constituents of the nutraceutical compositions of the present invention are present in an effective proportion and the nutraceutical composition is administered through an enteral feeding tube, the composition is effective to enhance absorption of nutrients, promote weight gain, remove an energy deficit or to increased appetite in a mammal such as a human or a horse. In mammals, especially humans or horses, presenting energy and nutrient deficits below the preferred healthy normal range, especially under stressful situations, the nutraceutical composition is also effective to provide energy from carbohydrates, rather than fat.

Energy in the normal healthy horse can and should be supplied to a large extent by fat. Fats (oils) are the densest forms of dietary energy, providing approximately 9 calories per gram. Energy density is very important when designing an enteral nutrition program. However, in most critical care cases the energy requirements should be met primarily by carbohydrates. Even though carbohydrates only provide approximately 4 calories per gram, there are reasons for carbohydrates to be chosen preferentially over fats in order to provide energy in an enteral form, or any tube feeding.

Mammals, such as horses, that do not receive enough energy in the form of carbohydrates will begin to catabolize muscle tissue and mobilize fat stores. This will result in a wasting syndrome clinically. Protein will be used for energy at the expense of anabolic processes. The by-products of this catabolic process (primarily urea and creatinine) place an extra workload on the liver and kidneys. This can be life threatening in many cases. Fat mobilization can result in a fatty liver syndrome in anorexic overweight mammalian patients, especially equines such as ponies. Therefore, adequate carbohydrate intake results in the two benefits of protein sparing and decreased hepatic lipidosis. Adequate carbohydrate intake should be provided early in order to prevent the catabolic process, and worsening conditions which becomes difficult to stop as time goes by.

In certain subjects, a dense carbohydrate source should be provided taking into consideration the hazards associated with too much too soon (laminitis and colic). Grain diets may not provide adequate carbohydrate due to the fact that the carbohydrate must be extruded in the digestion process. In critically ill patients, carbohydrate sources that require minimal processing are the most preferred way to meet energy needs in patients that are already in a negative energy state at presentation. Moreover, in the case of a horse experiencing hepatic compromise, energy requirements should be addressed through the addition of a low fat enteral feeding program.

When supplying protein requirements, consideration must be given to quality as well as quantity. Without high quality protein, a normally adequate quantity can still result in a mammal that is protein starved. This will lead to continued catabolism of muscle to meet the protein needs, especially in the face of disease. High quality protein also reduces the amount of work required by the liver and kidneys, which is critical in many disease states. Amino acid (AA) content and digestibility of the protein determines the quality of a protein. Certain amino acids are considered rate limiting such as lysine and threonine. Accordingly these must be supplied in the enteral diet or through a tube. Other amino acids such as arginine, carnitine and glutamine contribute to glycogenisis. Arginine is also capable of accelerating wound healing and inhibiting the development of neoplasia. Carnitine, which is not an essential amino acid, stimulates protein synthesis in the face of stress and may accelerate fatty-acid oxidation, which lowers lactic acid production. Carnitine has also been shown to reduce hepatic fat in several species. Glutamine is a non-essential amino which acts as an important energy source for the enterocytes of the entire intestinal tract and renal cells. In humans it has been postulated that during periods of stress glutamine serves as the primary precursor for glutathione, a powerful antioxidant. Moreover, glutamine shortens recovery periods in surgery and critically ill patients. Supplementation of the enteral feeding program with individual amino acids can be therapeutic in a wide variety of disease states.

Fiber is the hardest type of nutrient to supply a mammal such as a horse through a feeding tube. A reasonable goal for an enteral program is 25% of estimated need. Fiber is not soluble, so the particle size must be small enough to be suspended in the liquid meal and pass through the delivery system. As much fiber as possible needs to be given to provide mechanical stimulation of the digestive tract.

Vitamins are organic nutrients that are essential for normal metabolism. Well-balanced diets contain enough vitamins so that deficiencies are not as common as in the past. However, in the case of the critically ill equine, patients may be in need of vitamin supplementation in order to replace depleted vitamins, overcome ingredient deficiencies, or to address increased metabolic demands. Supplemental sources should be critically evaluated for quality as well as quantity.

Though often overlooked, minerals and trace minerals are extremely important in the critically ill patient. Minerals act as cofactors for enzymes for almost every reaction in the body. Everything from immune system function, to bone density, to protein, fat and carbohydrate metabolism is affected by mineral deficiencies. Mineral supplementation should be critically evaluated. Preferably, minerals and trace minerals are amino acid chelated minerals, due to the increased bioavailability of these formulations. Other organic complexes (citrates, gluconates, and lactates) have a higher biological value than the inorganic complexes (oxides, carbonates) with sulfates being in the middle. Unless minerals are supplied in the correct balance and form, minimal benefit will be obtained by supplementation.

The nutraceutical composition of the present invention includes a low fat component. The low fat component includes at least one of the following constituents: a protein component, a nutrient component, a functional food component, or a feed component. When any constituent is provided, that constituent is present in an effective proportion such that, when administered to a mammal in need thereof in an effective amount, the nutraceutical composition is effective to enhance nutrition. Such enhanced nutrition may improve absorption of nutrients, provide weight gain, remove an energy deficit or increase the appetite of a mammal. The preferred low fat component has a very low fat content below 3% by weight (wt.), preferably between 2% to 2.5% by wt., and even more preferably below 2.5% by wt.

In some preferred embodiments, the nutraceutical composition of the present invention includes a protein component. The purpose of the protein component is to provide energy to a mammalian subject in need thereof. The protein component of the present invention includes a bulking agent constituent, which typically makes up a large portion of the component, e.g., 99 wt. % or more of the total weight of the protein component. Examples of suitable bulking agents include protein-based materials, minerals and related mixtures.

In some embodiments, whey powder is the preferable bulking agent of the protein component for it has a low fat content and high protein content. Whey powder is typically a mixture of materials including, but not limited to lactose, protein, lactic acid and ash, and is one example of complex mixture that can be used as bulking agent. Whey (not whey protein concentrate) is a useful ingredient in an enteral feeding programs of mammals.

In some embodiments of the protein component, where whey flower is provided with lactose, lactase is provided and considered to be part of the bulking agent. It is advantageous to include lactase to break down the lactose in the solution prior to enteral feeding to ensure ease of digestibility. Where the protein component uses whey flower the composition commonly includes at least about 1% to about 5 wt. % of lactase. For example, a protein component may be made by adding 4 g of lactase (5000 IFCC/g) per 0.75 pound of whey powder.

In certain embodiments, whey ranges from 60-70% lactose and 7-11% protein. Lactase, such as a generic 9000 IFCC unit lactase tablet, should be added to the whey to produce equal amounts of glucose and galactose from the lactose. Both of glucose and galactose are very good sources of carbohydrate energy and are readily absorbed high in the small intestines. Glucose and galactose have different absorption rates so there is an advantage over feeding straight table sugar. Since absorption occurs high in the digestive tract, excess fermentation or colic do not occur when using this ingredient. The protein found in whey is of extremely high biological value. Whey is easy for the body to digest and utilize and effective as a protein source in the face of very depressed hepatic function, elevated blood ammonia levels, and hepatic encephalopathy. Whey is completely soluble in water. Whey is also only 1% fat, which is optimal when providing predetermined nutraceutical compositions of the present invention.

In order to determine the amount of lactase to add to the bulking agent of the present invention it may be necessary to estimate the lactase activity. One of ordinary skill in the art may readily use the following calculation to determine the amount of lactase need.

a. Start with generic 9000 IFCC unit lactase tablet.

b. 1 IFCC unit of lactase will break 1 micromol of lactose/min at 37 degrees C. at pH 4.5.

c. 24 hours*60 min=1440 micromols/d/unit

d. 1.44 mmol/d/unit*9000 units=12960 mmol/d/tablet MW of lactose=12*C+22*H+11*O=72+22+88=182 MW

e. 13 mol/d*182 MW=2366 g/d lactose to glucose and galactose

f. 2366 g/61% (where 61% is the lactose content of this whey)=3879 g of whey in 24 hours per tablet.

In some embodiments of the present invention the protein component may be prepared by incubating 500 g of whey in 1 gallon of water for 24 hours at room temperature with one lactase tablet. Each doubling of the lactase amount will cut the incubation time in half.

Although any size whey powder may be used to supplement a mammal, the size of the whey powder is of particular importance in enteral feeding. The whey powder should be fine to ease digestibility and to ensure uniform mixture when added to water. Preferably the whey powder is extra grade whey powder where the particle size is at least about 40 mesh using a nominal standard US sieve, in certain embodiments. 40 mesh means that at least about 95% of the whey powder passes through a 40 mesh nominal standard US sieve.

In some embodiments, the bulking agent in the protein component typically includes a carbohydrate component. The carbohydrate component can be a simple sugar (a monosaccharide), a disaccharide, a more complex carbohydrate or a mixture thereof. Specific examples of suitable saccharide to provide energy include glucose and galactose.

In certain embodiments the protein component is whey based and comprises between about 8-20% protein, preferably about 12% protein in some embodiments. Optionally, the protein component further comprises between about 60% to about 80% carbohydrate constituent, preferably 73% in some embodiments. The carbohydrate constituent is a mixture of glucose and galactose. In some embodiments, the protein component has a very low fat content below 2% by wt, preferably below 1.7%, and even more preferably below 1.5%.

In some embodiments, protein component is provided in dry powder form. Preferably the dry powder form is separated into packets for convenience and dosing. Typically 340 g of dry powder is portioned into one pack. In enteral feeding an adult horse is provided with 2 packets or about 680 g of protein component per adult serving.

Protein component may be prepared by adding two packets or about 680 g to water to form a slurry. Two packets are completely soluble in q.s. 1 gallon warm water. At no time should the water be hotter than 100 degrees Fahrenheit. The slurry should incubate at room temperature or greater for one hour in order to allow enough time for the lactase to break down the lactose. Next, the slurry should be refrigerated until use. Unused potions should be discarded after 24 hours.

An adult horse, such as a 1000 pound horse, should be provided three servings per day (6 packets or approximately 2040 g of protein component) which provides approximately 4263 Kcal energy and the equivalent 245 g protein. This yields 45% maintenance protein and 31% maintenance calories for a horse in need of nutritional support. The protein component is preferably absorbed in the proximal small intestines of the horse. Protein component is low in fat as described above and is suitable for administration to hypoglycemic newborns.

In some preferred embodiments, the nutraceutical composition of the present invention includes a nutrient component. In certain embodiments the nutrient component of the present invention includes at least one nutrient capable of promoting nutrition in a subject in need thereof such as an under or mal nourished horse. Preferably, in some embodiments, nutrient component is a mixture of various nutrients. Suitable nutrients for some embodiments of the nutrient component include at least one vitamin, mineral, trace mineral or antioxidant.

In certain embodiments, suitable vitamins for the nutrient component include vitamins categorized into two distinct classes: water soluble and fat soluble. Water soluble vitamins are not stored in the body; they are excreted if not utilized soon after ingestion. Fat soluble vitamins (Vitamins A, D, E, and K) are stored and have more of a potential for toxicity. These vitamins should be present in nutritionally significant amounts. Examples of vitamins which may be included in certain embodiments of the nutrient component of the present invention include vitamin A, vitamin B-1, vitamin B-2, vitamin B-3, vitamin B-6, vitamin B-12, vitamin C, vitamin D-3, vitamin E, vitamin K, biotin, choline, folic acid, and/or combinations of these.

In certain embodiments, the addition of individual vitamins is not recommended for they may be costly. General supplements are more economical and are usually closer to a proper balance than a collection of individual vitamins. Vitamin supplements usually also include minerals. When nutrient component of the present invention is a supplement or nutrient mixture it preferably contains all vitamins and minerals considered to be essential in the daily diet, in certain embodiments.

In certain embodiments, suitable mineral for the nutrient component include both minerals and trace minerals. Again, a quality general supplement may be utilized to provide a wide variety of minerals, trace minerals, and vitamins. Commercial equine supplements are available in powder form and most are at least suspendable in an enteral program. The quality level of the mineral components is often marginal though. Grinding tablets, opening capsules, or utilizing a human liquid supplement may be economical alternatives when the amount of bioavailable nutrients provided is considered. Electrolyte mineral levels should be monitored via blood work, and individual supplementation, either oral or by injection may be required.

In certain preferred embodiments, the nutrient component comprises one or more minerals such as calcium, magnesium, potassium, boron, molybdenum, vanadium and combinations of these. Moreover in certain preferred embodiments, the nutrient component comprises one or more trace minerals such as iron, copper, zinc, manganese, chromium, iodine, selenium, and combinations thereof. As used herein, these minerals are considered trace minerals. Preferably, all minerals or trace minerals are to be provided in an amino acid chelate form.

In certain embodiments, suitable constituents of the nutrient component include antioxidants. Vitamins, minerals, and other molecules which act in the body to scavenge oxygen radicals are antioxidants. Most patients that need nutritional support will benefit from a high level of dietary antioxidants. Preferably, in certain embodiments, antioxidants should be supplemented as a group instead of high levels of individual ones, due to the fact that any antioxidant can act as a pro-oxidant if present in high enough amounts.

Suitable antioxidants for use in certain embodiments of the present invention include using one or more antioxidant constituent such as CoQ10, pantothenic acid, DMG, grape seed extract, bioflavinoid, inositol, PABA, citrus bioflavonoid, pyctogen, and combinations of these.

In certain embodiments, suitable constituents of the nutrient component include amino acid. There are oral veterinary products that supply a combination of purified amino acids in a dextrose solution. For individual amino acids in significant amounts, health food stores may be the most convenient supply option. Equine supplement manufactures market a variety of products that may be utilized in the nutrient component of the present invention that provide amino acids. Suitable amino acid for use in certain embodiments of the present invention include using one or more amino acid constituents such as alanine, arginine, aspartic acid, cystine, glutamic acid, proline, glycine, histidine, hydroxyproline, isolcucine, leucine, lysine, methionine, phenylalanine, serine, threonine, tryptophan, tyrosine, valine, and combinations of these.

The nutrient component of the present invention may include addition inactive ingredients that have a variety of functions. These inactive ingredients may acts as, among other things, delivery system, filler, binder, or stabilizer for the active ingredients. Suitable inactive ingredients for certain embodiments of the nutrient component include: a delivery system consisting of water glycerine, custom trace mineral mix, citric acid, xanthium gum, natural flavors, stevia extract, and potassium sorbate as a stabilizer.

In some preferred embodiments, the composition of the present invention includes a functional food component. In certain embodiments the functional food component will include one or more of the following constituents: glucosamine, salt, amino acid, yeast, carnitine, fermentation extract, and combinations of these.

In certain embodiments the functional food component may include fermentation extracts including one or more prebiotic, probiotic, or synbiotic ingredients, or combinations of these. Ingredients of this type are very important to include in a nutritional support program. When normal dietary intake is interrupted, changes in the balance of normal digestive tract flora occur. Especially when the intake of fiber is reduced, support of the beneficial bacterial species is vital to the health and function of the digestive tract, in certain embodiments.

Prebiotic ingredients when provided to the digestive tract selectively support the growth of beneficial bacterial species over pathogenic ones. Prebiotics do not directly colonize the digestive tract. Prebiotics include yeast, yeast cultures, fungal cultures, and preferably, certain fibers (FOS-fructooligosaccharides). Probiotics are the actual bacterial species that, when introduced to the digestive tract actually colonize and produce beneficial effects. Preferably probiotics of the composition include Lactobacillus and Bifido. Synbiotics are products that contain both prebiotic and probiotic ingredients. Ingredients of this type are important to include in a nutritional support program. As used herein, synbiotic refers to a prebiotic and probiotic blend for gastrointestinal support. The synbiotic of the present composition is preferably a prebiotic and probiotic blend of Lactobacillus and Bifido, and FOS-fructooligosaccharides. Although various amounts of these ingredients may be combined in a mixture, supplied blends are available from distributors of these substances. Suitable blends for preferred embodiments of the present invention include probiotics from UAS Laboratories (product name is UAS Probiotic Blend), including the ingredients Bifidobacterium longum and Lactobacillus acidophilus, rice starch and fructooligosaccharides. The potency is over 10 billion Colony Forming Units per gram at the time of manufacture. This product may be blended with other constituents of the present invention. Preferably, the fermentation extract of the present invention is Amaferm brand fermentation extract.

In certain embodiments of the functional food component where glucosamine is used, glucosamine is provided as a chemical consisting of glucosamine sulphate, glucosamine sulfate 2KCL, glucosamine sulfate NaCl, glucosamine hydrochloride, N-acetylglucosamine, Poly-Nag. glucosamine, and combinations of these. In certain embodiments, N-acetyl-D-glucosamine is preferred. Supplementation with N-acetyl-glucosamine may help firm up the structural matrix of the intestinal tract. Though glucosamine appears to be highly absorbable, N-acetyl-glucosamine is directly incorporated into the intestinal mucosa and is not absorbed when provided orally. This improves the overall health of the intestinal tract under stress thereby contributing to its healing and increased absorption of other nutrients.

When the constituent of the functional food component is salt, preferably the salt is sodium chloride (NaCl). Sodium chloride requirement in an adult horse is approximately 30 grams per day (approximately 1 tablespoon). 30 grams per day is the preferred dosage of salt, even though the other constituents or components in the predetermined composition will contain salt. Such a dose is optimal due to the high margin of safety and probable increased losses of salt in most cases requiring enteral nutrition.

When the constituent of the functional food component is amino acid, preferably, it is a mixture of one or more ingredients including L-Glutamine, L-arginine, carnitine, and combinations of these. As used herein, carnitine is considered an amino acid.

In some preferred embodiments, the composition of the present invention includes a feed component. The feed component may be any suitable feed component having low fat content, such as alfalfa, corn meal, or oats. In certain embodiments mixtures of alfalfa and corn meal provide an optimal feed component. Preferably, about 50% alfalfa is blended with 50% corn meal in certain embodiments.

In certain embodiments, pelleted commercial feeds may be used as a feed component for they provide the simplest method by which to deliver some enteral nutrition. This is due to the fact that as a feed it is formulated for balance and completeness. It provides a balance of protein, carbohydrate, some fat, vitamins, minerals as well as some fiber. Commercial feeds may be utilized, so long as the fat content remains below 3% by weight of the composition. Accordingly, vegetable oil should not be added to create a pellet-oil slurry in water that can be passed through a nasogastric (NG) tube. Pelleted feeds are nutrient dense and make meeting calculated daily needs relatively easy. Pelleted feeds must be pulverized dry in a kitchen blender and mixed with oil and water just prior to feeding. If liquid is mixed with the feed to long, the cellulose will swell making administration difficult. Pelleted feeds are less preferred for use with the present invention for they were not designed to be soluble or suspendible, may be time consuming and frustrating, and may contain too much fat. This is true even though they may be balanced for the average healthy horse. Even a complete feed may be inadequate for the critical patient and may not allow for easy assembly of a predetermined low fat component.

Liquid preparations are not preferred for use as a feed compont for compositions of the present invention, even though human health care products such as Ensure®, Osmolite®, and Vital HN® have been used with some success in equine enteral feeding programs. These are easily administered via a NG tube due to a relatively low viscosity and no particulate matter. These diets have very little fiber content and may be cost prohibitive due to the large volume that must be fed. Liquid diets should be evaluated closely in light of metabolic conditions, being careful to choose diets that are not contraindicated in certain disease states. Many liquid products rely heavily on fat as an energy source (9-30%). Since fat is contraindicated in many critical patients, the fat must be removed before a liquid preparation may be utilized with the present invention. Moreover, liquid preparations should are less preferred because some solids need to be added to the feeding program at the appropriate time to properly stimulate the digestive tract in many mammals.

In certain embodiments, ground hay may be used to provide a feed component. Moreover, fiber may be provided from several sources such as powdered fiber supplements for people (not economical), fresh grass clippings, grinding baled hay, soaking hay cubes, or commercially ground or mealed hays. Larger particle sizes are more physiologically valuable, but in this case must be sacrificed so that more total fiber may be reasonably delivered. Practitioners are able to deliver more with the commercially mealed hays than with other forms. Since the total amount will be limited, it is best to use the available source that is the most nutrient dense. Accordingly alfalfa meal is a preferred constituent of the present invention. Moreover, it is available in most areas.

In certain embodiments corn meal is a suitable constituent of the feed component. Corn meal should not be confused with wheat flour. It has been surprisingly found that the most economical way to add a feed component (considering particle size) is to use corn meal from the grocery store. This form of corn meal is fortified with vitamins and minerals, has a very small particle size, will not “paste-up” in solution, is packaged in convenient sizes and is cheap, accordingly corn meal is preferred. As long as an accurate estimation of need is used, the risk of excess fermentation and colic is limited. The same is not true of wheat flour. Even though flour is of a smaller particle size, it is the least preferable choice for an equine feeding program. Wheat is highly fermentable and signs of colic appear using relatively small amounts. Moreover, corn meal mix is not a preferred constituent of the feed component for it contains almost half wheat flour. Accordingly, it has been surprisingly advantageous to select corn meal as a constituent of the feed component, for corn meal is only approximately 1.5% fat. By leaving the fat out or having low fat formulations, the constituents permit the administration of the optimal predetermined composition to a mammal in need thereof.

In all embodiments of the present invention, oils should be excluded, minimized, or avoided even though oils are an excellent way to provide fat to the enteral diet. One cup (8 ounces) of vegetable oil contains the energy equivalent of 3.5 cups of corn or 6 cups of oats. Animal fats such as tallow are between 88-92 percent digestible. Plant sources of fats such as corn oil or soybean oil are up to 94 percent digestible by the equine digestive tract. Oils are a source of fatty acids which are essential in many metabolic processes. Oils such as safflower oil, olive oil, and canola oil have high contents of omega-3 and omega-6 fatty acids. Even though corn oil is the most cost effective way to add energy from fat into an enteral feeding program, it is not the preferred energy source for the embodiments of the present invention.

Administration of the compositions of the present invention may be done by any method of administration known in the art.

Initiation of feeding: Set amount of time needs to pass before enteral nutrition support is started on a mammal in need thereof. Some amount of support can be provided in almost all situations, except for complete GI obstruction or the presence of reflux through the nasogastric tube. In the case of the mechanically dysphagic patient, with no metabolic or GI concerns, 25% of maintenance can be provided the first day with an increase of 25% each day in the absence of colic signs. The rate of increase should be slowed according to metabolic and GI tolerances in other cases. GI surgery cases can be fed the completely soluble (no particulate matter) components of the program almost immediately post surgery.

Volume limits: An appropriately designed program can provide an adult equine patient (with normal hepatic function) full maintenance calories and 80-100% of maintenance protein at a very reasonable cost. These nutrients can be delivered in 2 feedings of 2 gallons each. This frequency and quantity can be easily done with few complications due to tube placement in both ambulatory and hospital settings. More frequent, smaller feedings are slightly preferred and can deliver extra nutrients, but are not necessary in most situations.

Solubility: Selection of highly soluble ingredients is very important to a successful enteral feeding program. A limited amount of suspendable (not soluble) material may be included. This should be limited to the fiber portion as much as possible so that the maximum amount of fiber is included. Depending on how fine a grind is used, approximately 2 pounds of grain and 2 pounds of roughage can be provided in 2 feedings of 2 gallons each. The other components of the program need to be highly soluble. The contents should be continuously agitated and can pass through a bilge pump, funnel and stomach tube easily, but may be a problem with the smaller stomach pumps.

Estimation of Needs and Amounts Delivered: Needs and amounts of compositions are readily determinable by one of ordinary skill in the art. Moreover, the National Research Council's (NRC) publication of the Nutrient Requirements for Horses has tables listing the required amounts of energy, protein, forage, vitamins, and minerals. The tables list different requirements for several age, weight, and reproductive situations. The requirements are listed in the same units as most guaranteed analysis for commercial feed components. The NRC also has tables of analysis for most common feed ingredients. If a guaranteed analysis is not available for the specific product or constituent, these tables provide a good estimate of the nutrient amounts being provided. One of ordinary skill in the art understands that NRC requirements are for animals and Human equivalents are also available such as RDA's, RDI's, and DV's.

For example the following formula is provided to calculate rations in a large mammal:

Given: Horse under 200 kilogram, lactating mare, foaling to 3 months category. Body Weight (BW) is in kilograms.

Estimation of Digestible Energy (DE) Requirements (Mcal of DE/d)

DE=(1.4+0.03BW)+(0.04BW*0.792)

Estimation of Crude Protein (CP) Requirements (g/d)

CP={(40*Mcal of DE/d)+[(0.04BW*0.021*1000)/0.65]}/0.55

Estimation of Calcium (Ca) Requirements (g/d)

Ca=(0.04BW)+[(0.04BW*1.2)/0.5]

Estimation of Magnesium (Mg) Requirements (g/d)

Mg=(0.015BW)+[(0.04BW*0.09)/0.4]

Furthermore, the NRC formulas used to develop the tables mentioned above are also available. Using the formulas will provide a much more accurate estimate of nutrient needs because the actual weight of the patient can be input. As an example, the formulas for several major nutrients for an under 200 kg lactating mare are readily available. The basic formulas for maintenance requirements are adjusted for age, sex, weight, growth rate, activity level, gestation, and lactation. The starting point for estimating electrolyte needs should be calculated using the formulas. However, since metabolic conditions greatly effect individual needs, laboratory analysis of blood samples will heavily influence the appropriate amount to deliver.

When calculating the amount of protein deficit in a feeding program, preferably a protein adjustment factor is included. The NRC formulas assume a protein digestibility of 55%. Most of the ingredients of an enteral support program are much more bioavailable than this. Single amino acids, amino acid complexes and special protein sources such as whey will be adjusted upward the maximum amount of a multiple of 1.8. The amount of calories provided via IV dextrose infusion should be calculated as amount of dextrose provided in grams*3.4 calories. Whenever possible, the guaranteed analysis of individual ingredients should be used to calculate their nutrient contribution.

The formulas mentioned above can be used in the most basic computer spreadsheet program to estimate daily nutrient requirements, daily nutrient intake, and the difference between the two. Spreadsheets may show the basic information needed to appropriately design an enteral feeding program for the equine patient and create a predetermined composition of the present invention. It is very easy to adjust the formulas to create new sheets for subsequent cases having different needs.

The compositions of the present invention are all suitable for treating mammals having special metabolic needs due to hepatic dysfunction, renal dysfunction, or digestive tract disease.

Hepatic dysfunction: High quality protein is essential in order to prevent increased levels of circulating ammonia leading to hepatic encephalopathy. Protein should contain branched chain amino acids to prevent further ammonia formation. Glucose should be provided through the diet in order to prevent the need for hepatic glucose synthesis. Dietary fat should be reduced to the lowest possible level to decrease hepatic lipidosis and the amount of processing the liver must perform to make energy available to somatic cells.

Renal dysfunction: Since horses excrete calcium via the kidneys, calcium levels should be monitored and adjusted accordingly in the enteral diet. As with hepatic disease protein should be of the highest biological value to prevent ammonia accumulation.

Digestive tract disease: Small intestinal disorders require more fiber to maximize large intestine fermentation along with highly digestible protein. Glutamine should be incorporated to meet increased energy needs of the enterocytes in diseased or stressed states. In large intestine disorders pre and probiotics are beneficial in re-colonizing depleted microbe numbers. Electrolytes should be carefully monitored with blood work in diarrhea cases and adjusted (both enterally and parenterally) accordingly. Horses with diarrhea may benefit from probiotics, glutamine and N-acetyl-glucosamine. Protein losing enteropathy should be aggressively addressed by providing large amounts of high biological value protein. Colic signs should be closely monitored as feedstuffs are reintroduced to the diseased equine digestive tract.

Having discussed the composition of the present invention, it will be more clearly perceived and better understood from the following specific examples. It should be understood that the composition of the present invention may be supplied in various components which may be mixed together, or used independently. The total program has a fat content of less than 3% so that it can be used in the face of compromised (or underdeveloped) liver function. In cases where hepatic encephalography the protein component, nutrient component, and functional food component below are very highly digestible and will contribute very little to the waste N load.

Below is a specification for preferred extra grade whey powder for use with the protein component of certain embodiments of the present invention.

### Extra Grade Whey Powder

| | Specification | |
|---|---|---|
| Chemical analysis | | |
| Moisture % | <5% | |
| Fat % | <1.5 | |
| Inhibitors | none detected | |
| Protein % | >11 | |
| Lactose % | >65 | |
| Particle size | 95 | thru #40 mesh |
| Ash % | 8 | |
| pH | >5.6 | reconstituted |
| color | cream color | |
| flavor | clean whey | |
| Solubility | <1.25 | ml |
| Titratable Acidity % | 16 | maximum |
| | | reconstituted |
| scorched particles | 15 | mg maximum |
| Microbiological | | |
| Count (per ml of G): | | |
| Standard plate | <30,000 | |
| Coliform Count | <10 | |
| Yeast and Mold | <10 | |
| Salmonella/Listeria | <10 | |
| Nutritional Information | | |
| Calories | 354.00 | |
| Calories from Fat | 9.00 | |
| Total Fat | 85 | g |
| Saturated Fat | 0.53 | g |
| Cholesterol | 22.00 | mg |
| Sodium | 876.00 | mg |
| Total Carbohydrates | 73.00 | g |
| Dietary Fiber | 0.00 | g |
| Sugars | 72.00 | g |
| Protein | 12.00 | g |
| Vitamin A | 64 | IU |
| Calcium | 594 | mg |
| Vitamin C | 3 | mg |
| Iron | 0.6 | mg |

Below is a specification for a nutrient component for use with certain embodiments of the present invention. The serving size is preferably 3 ounces for a large mammal such as a horse.

| | Amount | |
|---|---|---|
| Nutrient Component | per serving | |
| Calories | 0 | |
| Carbohydrates | 0 | |
| Fats | 0 | |
| Vitamin A (Betacarotene-D. Salina) | 45,000 | IU |
| Vitamin B-1 (Thiamine HCL) | 90 | mg |
| Vitamin B-2 (Riboflavin) | 90 | mg |
| Vitamin B-3 (Niacinamide) | 90 | mg |
| Vitamin B-6 (Pyridoxine HCL) | 90 | mg |
| Vitamin B-12 (Cyanocobalamin) | 1200 | mcg |
| Vitamin C (Ascorbic Acid) | 3000 | mg |
| Vitamin D-3 (Cholecalciferol) | 300 | IU |
| Vitamin E (di-Alpha-tocopherol) | 750 | IU |
| Vitamin K (Menadione) | 30 | mg |
| Biotin | 900 | mcg |
| Calcium (from citrate chelate) | 1500 | mg |
| Choline (as Bitartrate) | 90 | mg |
| Chromium (from polynicotinate) | 900 | mcg |
| Copper (from Amino Acid Chelate) | 3 | mg |
| CoQ10 | 5 | mg |
| Pantothenic Acid (d-Calcium Pantothenate) | 450 | mg |
| DMG (dimethylglycine) | 90 | mg |
| Folic Acid (Folate) | 1200 | mcg |
| Grape Seed Extract | 75 | mg |
| Bioflavinoids | 13.35 | mg |
| Inositol | 90 | mg |
| Iron (Chelate) | 4 | mg |
| Magnesium (from citrate chelate) | 900 | mg |
| Manganese (from Amino Acid Chelate) | 7.5 | mg |
| PABA | 30 | mg |
| Potassium (from citrate chelate) | 297 | mg |
| Boron (from Amino Acid Chelate) | 2 | mg |
| Selenium (from Amino Acid Chelate) | 100 | mcg |
| Zinc (from Amino Acid Chelate) | 39 | mg |
| Molybdenum (from Amino Acid Chelate) | 450 | mcg |
| Citrus Bioflavonoids | 90 | mg |
| Vanadium (from vanadyl sulphate) | 90 | mcg |
| Pyctogen (Pine Bark Extract) | 30 | mg |
| Amino Acid Blend | 120 | mg |

(Containing Alanine, Arginine, Aspartic Acid, Cystine, Glutamic Acid, Proline, Glycine, Histidine, Hydroxyproline, Isolcucine, Leucine, Lysine, Methionine, Phenylalanine, Serine, Threonine, Tryptophan, Tyrosine, Valine

Inactive Ingredients: a delivery system consisting of water glycerine, Custom Trace Mineral Mix, citric acid, xanthium gum, natural flavors, stevia extract, and potassium sorbate as a stabilizer.

Below is a specification for a functional food component for use with certain embodiments of the present invention. The serving size is preferably 4 scoops (135 grams) for a large mammal such as an adult horse.

Functional food component

| | |
|---|---|
| L-Glutamine | 30 g |
| Salt | 30 g |
| Yeast | 30 g |
| Amaferm | 20 g |
| N-Acetyl-D-Glucosamine | 10 g |
| L-Arginine | 10 g |
| Carnitine | 5 g |

Below is a specification for a feed component for use with certain embodiments of the present invention. The serving size is preferably 1 pound per every 500 pounds of body weight per feeding for a large mammal such as an adult horse. The maximum recommended dosage is six pounds of feed component per day in the average adult horse.

Feed component

| | |
|---|---|
| 50% | alfalfa meal |
| 50% | corn meal |

The following case studies were conducted with mammals. The unexpected results demonstrate the effectiveness of the treatment.

A 6 year old, 100 kg, American Miniature Horse presented to the Veterinary Teaching Hospital with a 3 day history of anorexia, depression, and ventral edema. She was nursing a healthy five week old foal at the time of presentation. Initial physical examination showed weakness, reluctance to move, elevated pulse and respiratory rate, and ileus. CBC and blood chemistry profile showed azotemia, hypocalcemia, metabolic acidosis, and elevated liver enzymes and total bilirubin. A serum triglyceride concentration was greater than 2000 mg/dl. Based on the above findings a diagnosis of hyperlipemia and hepatic lipidosis was made. Initial treatment consisted of intravenous polyionic fluids to correct the azotemia and provide maintenance fluids. Intravenous dextrose was provided to correct the negative energy balance. Subcutaneous heparin and insulin were given to treat the hyperlipemia. Despite aggressive medical therapy, the mare developed signs of hepatoencepalopathy including circling, muscle fasciculations and severe depression, corresponding to elevated blood ammonia. On day 2 of hospitalization, a nutrition consultation was performed. A three-stage ration was formulated based on the mare's requirements for digestible energy, crude protein, calcium, and magnesium for maintenance and lactation. The predetermined diet was low in fat and protein, but provided enough calories to decrease the utilization of body fat. The diet was delivered through a small-bore nasogastric tube every 2-4 hours. Each stage was given for 24 hours and by the third day of oral feedings the neurologic signs had disappeared and the serum triglyceride concentration had decreased to within normal range. The liver enzymes and total bilirubin were also decreasing. She began eating on day six of hospitalization and the enteral feedings were discontinued. She was discharged on day 8 after presentation with instructions to provide supplemental feedings for the next 3 weeks and to wean the foal to decrease the energy demands on the mare. A follow-up visit performed 4 weeks after discharge showed no significant abnormalities on physical examination and a continued decrease in the serum concentration of liver enzymes.

Other examples: In the clinical setting, component based feeding has been encouraging. Simple diarrheas have responded well to treatment with a combination of glutamine, pre- and probiotics, N-acetyl-glucosamine, arginine and carnitine. In one horse with diarrhea secondary to heavy parasitism response was seen within 12 hours of administration. This combination has been used in all ages from newborns to the geriatric. Hypoproteinemic and anemic cases have responded well with this combination along with the addition of whey (lactase incubation) and a liquid vitamin/mineral supplement. Administration to newborns that are hypoglycemic is very encouraging. Horses that are not hypophagic have ingested top dressed supplemental nutrients well. Some of the lower quantity nutrients may also be syringe fed. In yet another horse, an 800 kg warmblood gelding with no symptoms except facial and glossal paresis was provided with a composition of the present invention. This gelding was economically maintained until enough coordination returned so that he could eat normally. Preferred embodiments were adequate for this case.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A composition suitable for administration through a tube comprising an effective proportion of components such that upon administration to a mammal in need thereof, the composition is effective to improve absorption of nutrients, increase appetite, promote weight gain, or reduce calorie deficit, wherein the composition comprises less than 3% by weight fat.

2. The composition of claim 1 characterized as having between about 2% to about 2.5% fat by weight.

3. The composition of claim 1 comprising a feed component comprising ingredients selected from the group consisting of alfalfa, corn meal, oats, and combinations thereof.

4. The composition of claim 1 comprising liquid vitamin.

5. The composition of claim 1 comprising a nutrient component comprising at least one vitamin selected from the group consisting of vitamin A, vitamin B-1, vitamin B-2, vitamin B-3, vitamin B-6, vitamin B-12, vitamin C, vitamin D-3, vitamin E, vitamin K, biotin, choline, folic acid, and combinations thereof.

6. The composition of claim 1 comprising a nutrient component comprising at least one mineral selected from the group consisting of calcium, magnesium, potassium, boron, molybdenum, vanadium and combinations thereof.

7. The composition of claim 1 comprising a nutrient component comprising at least one trace mineral selected from the group consisting of iron, copper, zinc, manganese, chromium, iodine, selenium, and combinations thereof.

8. The composition of claim 1 comprising a nutrient component comprising at least one antioxidant selected from the group consisting of CoQ10, pantothenic acid, DMG, grape seed extract, bioflavinoid, inositol, PABA, citrus bioflavonoid, pyctogen, and combinations thereof.

9. The composition of claim 1 comprising a nutrient component comprising at least one amino acid selected from the group consisting of alanine, arginine, aspartic acid, cystine, glutamic acid, proline, glycine, histidine, hydroxyproline, isolcucine, leucine, lysine, methionine, phenylalanine, serine, threonine, tryptophan, tyrosine, valine, and combinations thereof.

10. The composition of claim 1 comprising whey powder.

11. The composition of claim 10 wherein the whey powder is smaller than about 45 mesh.

12. The composition of claim 1 comprising a protein component comprising the following constituents: whey powder, and lactase.

13. The composition of claim 12 wherein the constituents of the protein component are present in the following approximate effective proportions: between about 95 and about 100% by weight of whey powder, and between about 1-5% by weight of lactase.

14. The composition of claim 12 wherein the protein component further comprising at least one monosaccharide.

15. The composition of claim 14 wherein the monosaccharide is selected from the group consisting of glucose, galactose, fructose, and combinations thereof.

16. The composition of claim 1 comprising a nutrient component comprising ingredients selected from the group consisting of vitamin, mineral, trace mineral, antioxidant, amino acid and combinations thereof.

17. The composition of claim 16 wherein the mineral or trace mineral is in amino acid chelate form.

18. The composition of claim 16 wherein the amino acid is selected from the group consisting of L-glutamine, L-arginine, carnitine, and combinations of these.

19. The composition of claim 1 comprising a functional food component comprising at least one ingredient selected from the group consisting of glucosamine, salt, amino acid, yeast, fermentation extract, and combinations thereof.

20. The composition of claim 19 wherein glucosamine is a chemical selected from the group consisting of glucosamine sulphate, glucosamine sulfate 2KCL, glucosamine sulfate NaCl, glucosamine hydrochloride, N-acetylglucosamine, Poly-Nag. glucosamine, and combinations thereof.

21. The composition of claim 19 wherein the salt is sodium chloride.

22. The composition of claim 19 wherein the fermentation extract further comprises a prebiotic, probiotic, synbiotic, or combinations thereof.

23. The composition of claim 1 comprising the following components:
a protein component,
a nutrient component,
a functional food component, and
a feed component, wherein each component is present in an effective proportion such that, when administered to a mammal in need thereof in an effective amount, the nutraceutical composition is effective to improve absorption of nutrients.

24. The composition of claim 23 in a liquid dosage form.

25. A method for reducing energy deficit in a mammal comprising the step of enterically administering to the mammal an energy promoting effective amount of a composition having less than 3% fat comprising an effective proportion of components.

26. The method of claim 25 wherein the composition comprises between about 2% to about 2.5% fat by weight.

27. The method of claim 25 wherein the composition comprises a nutrient component further comprising at least one ingredient selected from the group consisting of vitamin, mineral, trace mineral, antioxidant, amino acid and combinations thereof.

28. The method of claim 25 wherein the composition comprises a nutrient component further comprising liquid vitamin.

29. The method of claim 25 wherein the composition comprises a nutrient component comprising at least one vitamin selected from the group consisting of vitamin A, vitamin B-1, vitamin B-2, vitamin B-3, vitamin B-6, vitamin B-12, vitamin C, vitamin D-3, vitamin E, vitamin K, biotin, choline, folic acid, and combinations thereof.

30. The method of claim 25 wherein the composition comprises a nutrient component comprising at least one antioxidant selected from the group consisting of CoQ10, pantothenic acid, DMG, grape seed extract, bioflavinoid, inositol, PABA, citrus bioflavonoid, pyctogen, and combinations thereof.

31. The method of claim 25 wherein the composition comprises a feed component further comprising at least one ingredient selected from the group consisting of alfalfa, oats, and combinations thereof.

32. The method of claim 25 wherein the composition is in an oral liquid dosage form, or a powder form.

33. The method of claim 25 wherein said mammal is a human, horse, dog, cow, pig, goat, or sheep.

34. The method of claim 25 wherein the composition comprises a nutrient component comprising at least one mineral selected from the group consisting of calcium, magnesium, potassium, boron, molybdenum, vanadium and combinations thereof.

35. The method of claim 34 wherein the mineral is in amino acid chelate form.

36. The method of claim 25 wherein the composition comprises a nutrient component comprising at least one trace mineral selected from the group consisting of iron, copper, zinc, manganese, chromium, iodine, selenium, and combinations thereof.

37. The method of claim 36 wherein the mineral is in amino acid chelate form.

38. The method of claim 25 wherein the composition comprises at least one ingredient selected from the group consisting of whey powder, lactase, and combinations thereof.

39. The method of claim 38 wherein the composition comprises whey powder and lactase, wherein the whey powder and lactase are present in the following approximate effective proportions: between about 95% to about 100% by weight of whey powder, and between about 1% to about 5% by weight of lactase.

40. The method of claim 38 wherein the whey powder is smaller than about 45 mesh.

41. The method of claim 38 wherein the composition further comprises at least one monosaccharide.

42. The method of claim 41 wherein the monosaccharide is selected from the group consisting of glucose, galactose, fructose, and combinations thereof.

43. The method of claim 25 wherein the composition comprises a nutrient component comprising at least one amino acid selected from the group consisting of alanine, arginine, aspartic acid, cystine, glutamic acid, proline, glycine, histidine, hydroxyproline, isolcucine, leucine, lysine, methionine, phenylalanine, serine, threonine, tryptophan, tyrosine, valine, and combinations thereof.

44. The method of claim 25 wherein the composition comprises a functional food component further comprising at least one ingredient selected from the group consisting of glucosamine, salt, amino acid, yeast, fermentation extract, and combinations thereof.

45. The method of claim 44 wherein the glucosamine is a chemical selected from the group consisting of glucosamine sulphate, glucosamine sulfate 2KCL, glucosamine sulfate NaCl, glucosamine hydrochloride, N-acetylglucosamine, Poly-Nag. glucosamine, and combinations thereof.

46. The method of claim 44 wherein the salt is sodium chloride.

47. The method of claim 44 wherein the amino acid is selected from the group consisting of L-glutamine, L-arginine, carnitine, and combinations of these.

48. The method of claim 44 wherein the fermentation extract comprises at least one ingredient selected from the group consisting of prebiotic, probiotic, synbiotic, and combinations thereof.

49. A method of treating a mammal comprising the step of enterically administering to the mammal a composition suitable for administration through a tube comprising an effective proportion of components such that upon administration to a mammal in need thereof, the composition is effective to improve absorption of nutrients, increase appetite, promote weight gain, or reduce calorie deficit, wherein the composition comprises less than 3% by weight fat.
